(19) United States
(12) Patent Application Publication
(10) Pub. No.: US 2013/0345634 A1
(43) Pub. Date: Dec. 26, 2013

(54) DELIVERY DEVICES, SYSTEMS AND METHODS FOR DELIVERING THERAPEUTIC AGENTS

(71) Applicants: CELL PRECISION, LLC, Atlanta, GA (US); EMORY UNIVERSITY, Atlanta, GA (US)

(72) Inventors: Michael L. Wach, Alpharetta, GA (US); Raymond P. Vito, Atlanta, GA (US); W. Robert Taylor, Stone Mountain, GA (US); Jack Griffis, Decatur, GA (US)

(21) Appl. No.: 13/924,698

(22) Filed: Jun. 24, 2013

Related U.S. Application Data
(60) Provisional application No. 61/663,731, filed on Jun. 25, 2012.

(51) Int. Cl. A61M 25/06

(52) U.S. Cl. CPC A61M 25/0606
USPC 604/164.01

(57) ABSTRACT

Delivery devices, systems, and methods are configured to increase the retention of the therapeutic agent, and thereby increase the dose of the agent delivered. The delivery device may include a delivery body and a retractable member configured to move with respect to the delivery body. The retractable member may include a puncture member configured to create a delivery channel at the treatment site. The body may include an opening through which an agent may be delivered after the retractable member has been retracted within the device.

### CROSS REFERENCE TO RELATED APPLICATION

This application claims priority to Provisional Application Ser. No. 61/663,731 filed Jun. 25, 2012, which is hereby incorporated by reference in its entirety.

### BACKGROUND

An emerging approach to treating disease entails delivering a therapeutic agent at the treatment site and focuses the therapeutic agent's effect on the diseased tissue.

Conventional technologies for delivering such agents, generally lack sufficient sophistication for realizing full therapeutic potential. Aggressive handling resulting from these technologies, such as attempting to inject the agents with a conventional syringe, may compromise therapeutic effectiveness due to shear forces, imprecise placement at a treatment site, and leakage or escape of the agents from the treatment site after the delivery. Accordingly, a need exists for improved delivery methods and devices.

### SUMMARY

The disclosure relates to delivery devices, systems, and methods for delivering a therapeutic agent. These devices, systems, and methods may improve the reliability of placement and reduce the likelihood of leakage of the agents from the treatment site after the delivery of the agents. These devices, systems, and methods, may also reduce or ore eliminate shear stress associated with the delivery of an agent.

In some embodiments, the disclosure may relate to a device for delivering an agent to a treatment site. The device may include a body having a first end, an opposing second end and a length between the first end and the second end, the body including a channel between the first end and a second end, the channel including at least one opening configured to deliver the agent, the body including a first section and a second section, a diameter of the first section being larger than a diameter of the second section, a portion of the second section being parallel with the first section, the diameter of the inner channel corresponding to the diameter of the body. The body may also include a retractable member having a first end, an opposing second end, and a length between the first end and the second end, the retractable member including at least one puncture member configured to penetrate tissue to create a delivery channel at the treatment site, the at least one puncture member being disposed at the second end, the retractable member being configured to move relative to the body. The retractable member may be configured to move with respect to the body. The body may be configured to deliver the agent when the retractable member including the puncture member is retracted within the body. The retractable member may be configured to move axially and/or radially with respect to the delivery body.

In some embodiments, the second section may include a first portion and a second portion, the second portion being parallel with the first section. In some embodiments, the second portion may be longer than the first portion. In other embodiments, the first portion may be longer than the second portion. In some embodiments, the first portion may have a diameter that tapers from the first section to the second portion. In some embodiments, a portion of the second section being configured to support the delivery channel.

In some embodiments, the opening may be perpendicular to the length of the body. In other embodiments, the opening may be transverse to the length of the body. In some embodiments, an inner surface of at least a portion of the second section may be exposed when the retractable member is retracted. In some embodiments, at least a portion of the second section may be configured to support the delivery channel while delivering the agent.

In some embodiments, the retractable member may include a plurality of ports at least partially disposed on the second section. In some embodiments, the ports may be configured to disperse the agent when the retractable member is retracted. In some embodiments, the ports may be the same size. In other embodiments, the ports may be different sizes. In some embodiments, the retractable member may have a diameter configured to seal the ports so as to prevent leakage while the delivery device is being advanced.

In some embodiments, the retractable member may have a diameter configured to seal the opening of the delivery body.

In some embodiments, the puncture member may include at least one beveled edge. In some embodiments, the puncture member may include a plurality of beveled edges. In other embodiments, the puncture member may have a conical shape. In other embodiments, the puncture member may include a puncture tip. In some embodiments, the puncture tip may be disposed at an edge of the puncture member. In other embodiments, the puncture tip may be disposed at a center of the puncture member.

In some embodiments, the puncture member may include smooth edges and/or tip. In other embodiments, the puncture member may include a cutting edge.

In some embodiments, the retractable member may be solid. In other embodiments, the retractable member may be partially hollow. In some embodiments, the retractable member may be substantially rigid. In some embodiments, the retractable member may be at least partially flexible. In some embodiments, the retractable member may be tensioned in a predetermined direction.

In some embodiments, the device may be configured to deliver an agent to a treatment site. The device may include a body having a first end, an opposing second end and a length between the first end and the second end, the body including a channel between the first end and a second end, the channel including at least one opening configured to deliver the agent, the body including a first section and a second section, the second section including a first portion and a second portion, a diameter of the first section being larger than a diameter of the second section, the second portion being parallel with the first section, the diameter of the inner channel corresponding to the diameter of the body. The device may further include a retractable member having a first end, an opposing second end, and a length between the first end and the second end, the retractable member including a first puncture member configured to penetrate tissue to create a delivery channel at the treatment site, the first puncture member being disposed at the second end, the retractable member being configured to move relative to the body. The retractable member may be configured to move between at least a first position and a second position, the first position corresponding to when a portion of the retractable member is disposed within the channel, the second position corresponding to when at least the portion of the retractable member is retracted within the body. The delivery body may be configured to deliver the agent when the retractable member is disposed at the second position.

In some embodiments, the body may include a plurality of extending members disposed at the second end, the body being configured to move between a tension state and an untension state. In some embodiments, when in an untensioned state, the extending members may be configured to expand or dilate the delivery channel. In some embodiments, the extending members may have a width that tapers from the first end toward the second end. In some embodiments, the body may include a plurality of spaces, each space being between a pair of extending members. In some embodiments, the body may include four extending members and four spaces that are evenly spaced.

In some embodiments, the retractable member may include a plurality of protruding members and curved surfaces between the protruding members, the protruding members being configured to overlap the extending members and the curved surfaces being configured to move under the protruding member.

In some embodiments, the body may include at least a second puncture member, the second puncture member being disposed along the length of the body. The second puncture member may include a cutting edge configured to cut tissue and a tissue removal port configured to retain and remove the tissue.

In some embodiments, the retractable member may include an opening disposed at the puncture member and an inner channel that disposed along the length, the inner channel communicating with the opening.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows a delivery device according to embodiments;

FIG. 2 shows an enlarged view of the delivery device of FIG. 1;

FIG. 3 shows the delivery device of FIG. 1 in a different position;

FIG. 4 shows a delivery device according to embodiments in a state of operation;

FIG. 5 shows the delivery device of FIG. 4 in a different state of operation;

FIG. 6 shows the delivery device of FIG. 5 in a different state of operation;

FIG. 7 shows the delivery device of FIG. 6 in a different state of operation;

FIG. 8 shows the delivery device of FIG. 7 in a different state of operation;

FIG. 9 shows a delivery body according to embodiments;

FIG. 10 shows an enlarged view of the delivery body of FIG. 9;

FIG. 11 shows another view of the delivery body of FIG. 9;

FIG. 12 shows a cross-sectional view of the delivery body taken along the line 12-12 of FIG. 11;

FIG. 13 shows a delivery body according to embodiments;

FIG. 14 shows a delivery device according to embodiments;

FIG. 15 shows a side view of the delivery device of FIG. 14;

FIG. 16 shows a cross-sectional view of the delivery body taken along the line 16-16 of FIG. 15;

FIG. 17 shows a delivery body according to embodiments;

FIG. 18 shows a front view of the delivery body of FIG. 17;

FIG. 19 shows a cross-sectional view of the delivery body taken along the line 19-19 of FIG. 17;

FIG. 20 shows a delivery device according to embodiments;

FIG. 21 shows a delivery body according to embodiments;

FIG. 22 shows an enlarged view of the delivery body of FIG. 21;

FIG. 23 shows a top view of the delivery body of FIG. 21;

FIG. 24 shows a cross-sectional view of the delivery body taken along the line 24-24 of FIG. 23;

FIG. 25 shows another delivery device according to embodiments;

FIG. 26 shows an enlarged view of the delivery device of FIG. 25;

FIG. 27 shows a top view of the delivery device of FIG. 25;

FIG. 28 shows a cross-sectional view of the delivery device taken along the line 28-28 of FIG. 27;

FIG. 29 shows a side view of the delivery device of FIG. 25;

FIG. 30 shows an enlarged view of the delivery device of FIG. 29;

FIG. 31 shows a delivery body according to embodiments;

FIG. 32 shows an enlarged view of the delivery body of FIG. 31;

FIG. 33 shows a side view of the delivery body of FIG. 31;

FIG. 34 shows an enlarged view of the delivery body of FIG. 33;

FIG. 35 shows a front view of the delivery body of FIG. 31;

FIG. 36 shows a cross-sectional view of the delivery body taken along the line 36-36 of FIG. 33;

FIG. 37 shows an enlarged view of the delivery body of FIG. 36;

FIG. 38 shows a delivery device according to embodiments;

FIG. 39 shows an enlarged view of the delivery device of FIG. 38;

FIG. 40 shows a side view of the delivery device of FIG. 38;

FIG. 41 shows an enlarged view of the delivery device of FIG. 40;

FIG. 42 shows a front view of the delivery device of FIG. 38;

FIG. 43 shows a cross-sectional view of the delivery device taken along the line 41-41 of FIG. 40;

FIG. 44 shows an enlarged view of the delivery device of FIG. 43;

FIG. 45 shows the delivery device of FIG. 38 in an open or extended position;

FIG. 46 shows an enlarged view of the delivery device of FIG. 45;

FIG. 47 shows a side view of the delivery device of FIG. 45;

FIG. 48 shows a front view of the delivery device of FIG. 45;

FIG. 49 shows an enlarged view of the delivery device of FIG. 47;

FIG. 50 shows a cross-sectional view of the delivery device taken along the line 50-50 of FIG. 47;

FIG. 51 shows an enlarged view of the delivery device of FIG. 50;

FIG. 52 shows the delivery device of FIG. 38 in a partially retracted position;

FIG. 53 shows an enlarged view of the delivery device of FIG. 52;

FIG. 54 shows a side view of the delivery device of FIG. 52;

FIG. 55 shows a front view of the delivery device of FIG. 52;

FIG. 56 shows an enlarged view of the delivery device of FIG. 54;

FIG. 57 shows a cross-sectional view of the delivery device taken along the line 57-57 of FIG. 54;

FIG. 58 shows an enlarged view of the delivery device of FIG. 57;

FIG. 59 shows another delivery device according to embodiments;

FIG. 60 shows an enlarged view of the FIG. 59;

FIG. 61 shows a side view of the delivery device of FIG. 59;

FIG. 62 shows a cross-sectional view of the delivery device taken along the line 62-62 of FIG. 61;

FIG. 63 shows an enlarged view of the delivery device of FIG. 62;

FIG. 64 shows a delivery device, according to embodiments, in a closed or resting position;

FIG. 65 shows an enlarged view of the FIG. 64;

FIG. 66 shows a cross-sectional view of the delivery device taken along the line 66-66 of FIG. 64;

FIG. 67 shows an enlarged view of the delivery device of FIG. 66;

FIG. 68 shows the delivery device of FIG. 64 in an extended or open position;

FIG. 69 shows an enlarged view of the FIG. 68;

FIG. 70 shows a delivery device according to embodiments;

FIG. 71 shows an enlarged view of the FIG. 70;

FIG. 72 shows a cross-sectional view of the delivery device taken along the line 72-72 of FIG. 71;

FIG. 73 shows an enlarged view of the delivery device of FIG. 72;

FIG. 74 shows a delivery device according to embodiments;

FIG. 75 shows an enlarged view of the delivery device of FIG. 74;

FIG. 76 shows a side view of the delivery device of FIG. 74;

FIG. 77 shows a cross-sectional view of the delivery device taken along the line 77-77 of FIG. 76;

FIG. 78 shows an enlarged view of the delivery device of FIG. 77;

FIG. 79 shows the delivery device of FIG. 74 in a different, rotated position;

FIG. 80 shows an enlarged view of the delivery device of FIG. 79;

FIG. 81 shows a state of operation of a delivery system according embodiments;

FIG. 82 shows a delivery system according to embodiments; and

FIG. 83 shows an enlarged view of the system of FIG. 82.

### DETAILED DESCRIPTION OF THE EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications

It will be understood that the delivery devices according to the embodiments may be implanted into a patient with use of a delivery system. The delivery devices may be a part of the delivery system. The proximal end (not shown in detail) of the disclosed delivery devices may be attached or connected to the delivery system. The disclosed delivery devices may be either part of the main body of the delivery system or may be a detachable component. The delivery system may be any known delivery system configured to advance and control the advancement of the delivery device to the treatment site. The delivery system may include but is not limited to a handle assembly, an introducer, and a guidewire. The delivery systems are not limited to those shown in the figures and the delivery devices may be used with other delivery systems. It will also be understood that a clinician may use a delivery system to control the operation of the disclosed delivery devices. It will be further understood that although the operations of the disclosed delivery devices are discussed with respect to the actions of a clinician, delivery systems may be configured to perform these actions.

The delivery systems and the disclosed devices may be adjusted and sized for the anatomy and the direction of the desired point of insertion. For example, the delivery systems and devices may be sized for lengthwise insertion into a vascular lumen (e.g., 1.0-2.0 mm in diameter). The disclosed delivery systems and devices may also be adjusted according to the requirements of the therapeutic agent(s) to be delivered to the patient. The requirements may include, but not limited to, the size and the dosage amount of the therapeutic agent(s).

The delivery devices and systems according to embodiments may be configured to deliver a therapeutic agent(s). The “therapeutic agent” may include any one or more substance, compound, composition, formulation, and/or agent capable of exerting an effect, such as an therapeutic, prophylactic or diagnostic effect, on a patient. The therapeutic agent(s) may be embedded or dispersed in alginate strings or filaments, encapsulated, suspended in a highly viscous solution, suspended in a high-volume fraction solution (e.g., saline solution), as well as provided in any known vehicle to deliver a therapeutic agent. Such strings or filaments may be end loaded into the distal end of the delivery devices and systems according to embodiments, without necessarily feeding from the proximal end to the distal end. Suitable techniques for making therapeutic agent(s), for example, making strings and filaments comprising cells and alginate, are known in the art and may be readily created by those of ordinary skill having benefit of the present teaching. See, for example, “Grafting microcapsules of genetically modified cells: Therapeutic potential in spinal cord injury;” M. Wheatley, N. Dhoot, S. Kanakasabai and I. Fischer; Drexel University, Philadelphia, USA; XVth International Workshop on Bioencapsulation, Vienna, Au.; Sep. 6-8, 2007; S7-2, pages 1-4.

The terms “distal” and “proximal” used herein with respect to the delivery device and features are with respect to the position of the delivery device when in use. “Distal” indicates an end of the delivery device or a feature of the device closest to, or a direction towards the treatment site, and “proximal” indicates an end of the device or a feature of the device farthest from, or a direction away from the treatment site. “Treatment site” refers to any site or region of a subject, human or animal, intended to be treated, such as a tissue of an organ or muscle.

The delivery systems, devices and methods according to embodiments address potentially problematic levels of shear stress associated with delivering agents through conventional needles. Conventional needles may subject these problematic levels of shear stress when the agent travels through a needle's narrow internal channel. Additionally, the delivery systems, devices and methods according to embodiments address potentially problematic leakage or escape of therapeutic agents from the treatment site after the delivery of the agents by a conventional injection device. A conventional delivery device can form a puncture or needle track in a tissue after the device has been removed from the tissue after the injection of an agent. For example, an agent delivered into a tissue of an organ that undergoes expansion and contraction, such as the heart, can facilitate the leakage of previously-injected therapeutic agents from the organ tissue through the puncture track.

A delivery device according to embodiments may reduce or eliminate shear stress associated with the delivery of an agent because the structure of the delivery device is configured to reduce resistance during the delivery. Additionally, a delivery device according to embodiments may increase the retention of the therapeutic agent, and thereby increase the dose of agents delivered, because the delivery device is configured to create a delivery channel in the treatment site for the agent before or in advance of the delivery of the agents. A delivery device according to the embodiments thus may reduce the difficulties with delivering high viscosity therapeutic agents.

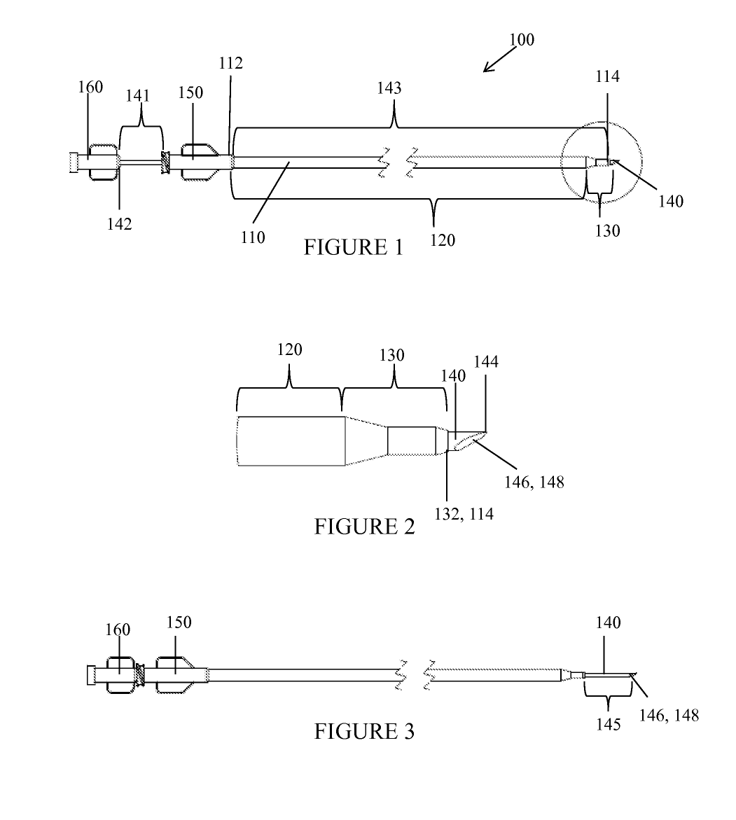

FIGS. 1 through 80 show examples of the delivery devices, configured to reduce shear and resistance on agents to be delivered, delivery bodies, and retractable members, and operation thereof, according to different embodiments. It will be understood that the delivery devices are not limited to the configuration and/or combination of the retractable member and the delivery body shown in the figures. The delivery devices may include any combination of the embodiments of the retractable member and the delivery body.

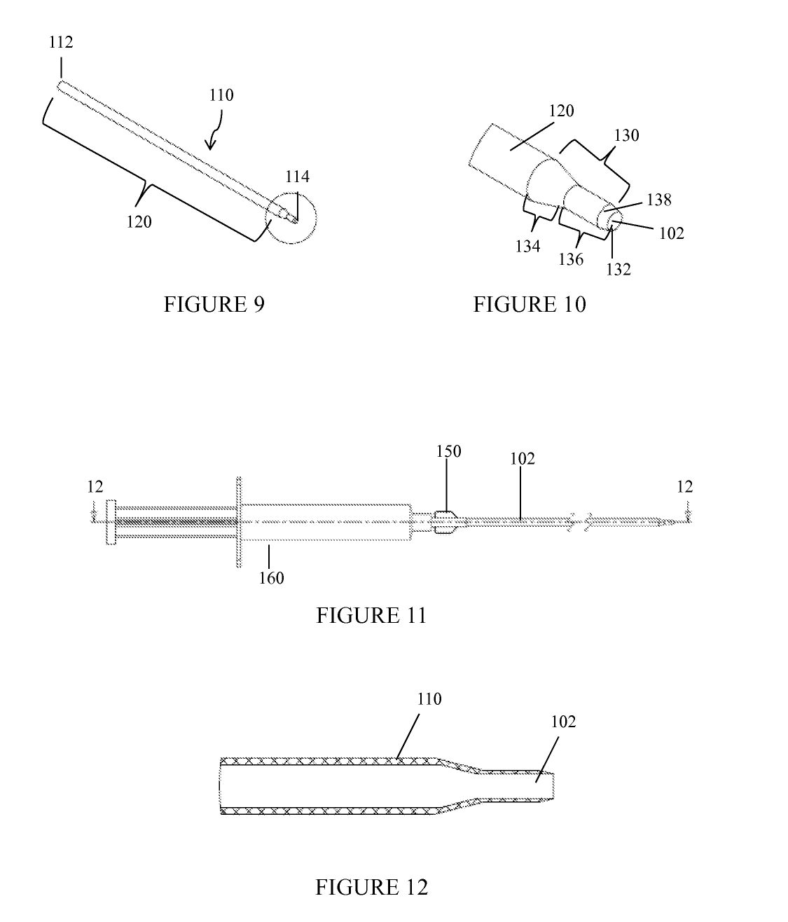

FIGS. 1 through 3 show a delivery device according to some embodiments. As shown in FIGS. 1 through 3, a delivery device 100 may include a delivery body 110. The delivery device 100 may also include a retractable member 140 configured to be movable with respect to the delivery body 110. In some embodiments, at least the retractable member 140 may be configured to create a delivery channel at or within a treatment site. In some embodiments, the retractable member 140, in combination, with a portion of the delivery body 110, may be configured to create a delivery channel at the treatment site. FIGS. 9 through 12 show the delivery body 110 without a retractable member.

In some embodiments, the delivery body 110 may include a first (proximal) end 112, an opposing second (distal) end 114, and a length therebetween. The delivery body 110 may include an inner channel or lumen 102 that extends along its length between the first end 112 and the second end 114.

In some embodiments, the delivery body may include an opening disposed at each of the ends. The delivery body 110 may include a delivery opening (also referred to as “opening”) 132. The opening 132 may be disposed at the second end 114. The opening 132 may be configured to deliver a therapeutic agent to a treatment site.

In some embodiments, the delivery body 110 may include at least two sections. The delivery body 110 may include a first section 120 and a second section (also referred to as “delivery throat”) 130.

In some embodiments, one, some, or all of the dimensions of the first section 120 and the second section 130 may be the same. In other embodiments, one, some, or all of the dimensions of the first section 120 and the second section 130 may be different. In some embodiments, for example, the first and second sections 120 and 130 may differ in length, diameter, shape, or a combination thereof. As shown in FIGS. 1 through 3, in some embodiments, the first section 110 may be longer than the second section 130.

In some embodiments, the first section 120 may have a larger diameter or bore than the second section 130. At least the diameter of the first section 120 and/or a portion of the second section 130 may be configured to control the positioning of the delivery device 100 with respect to the treatment site.

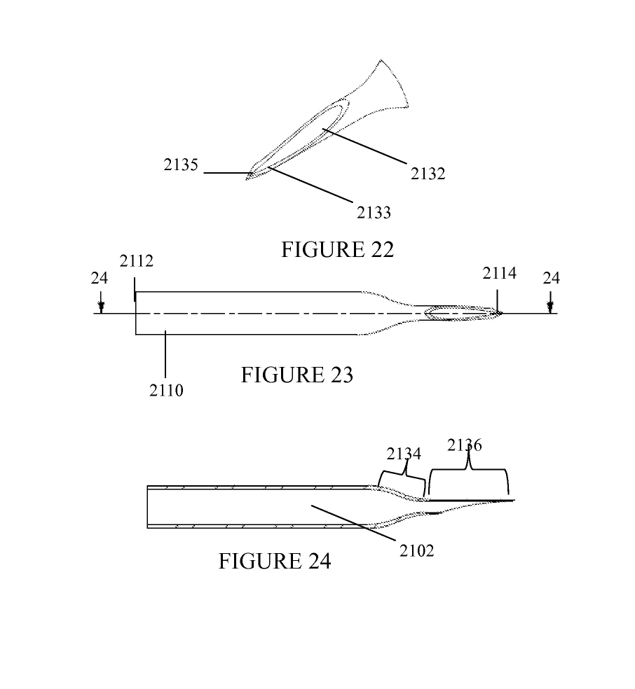

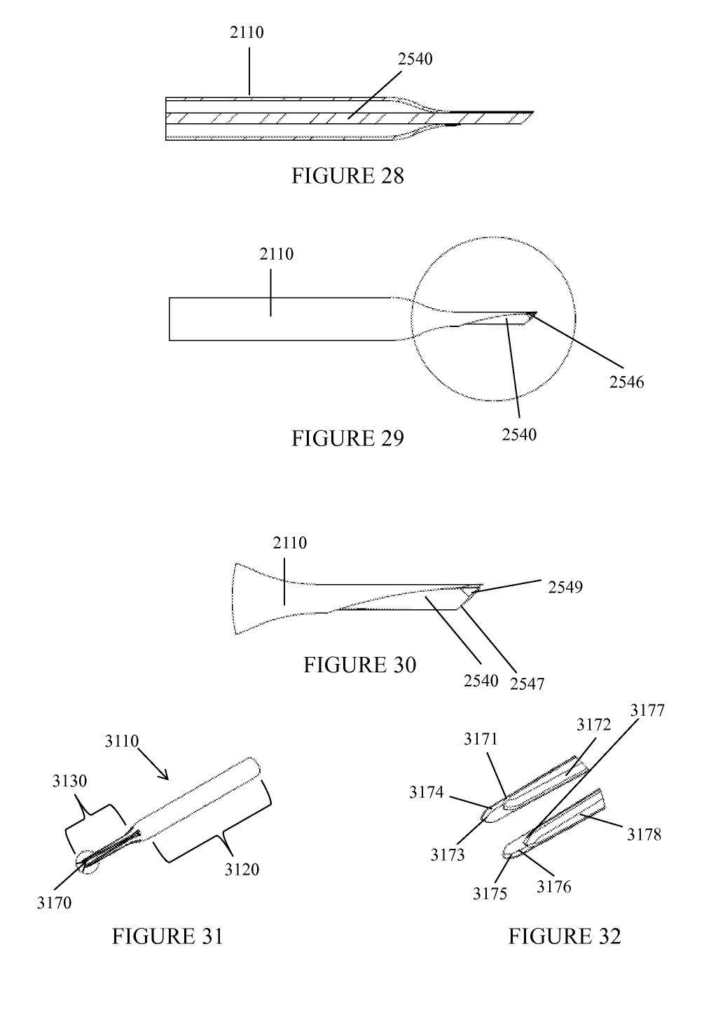

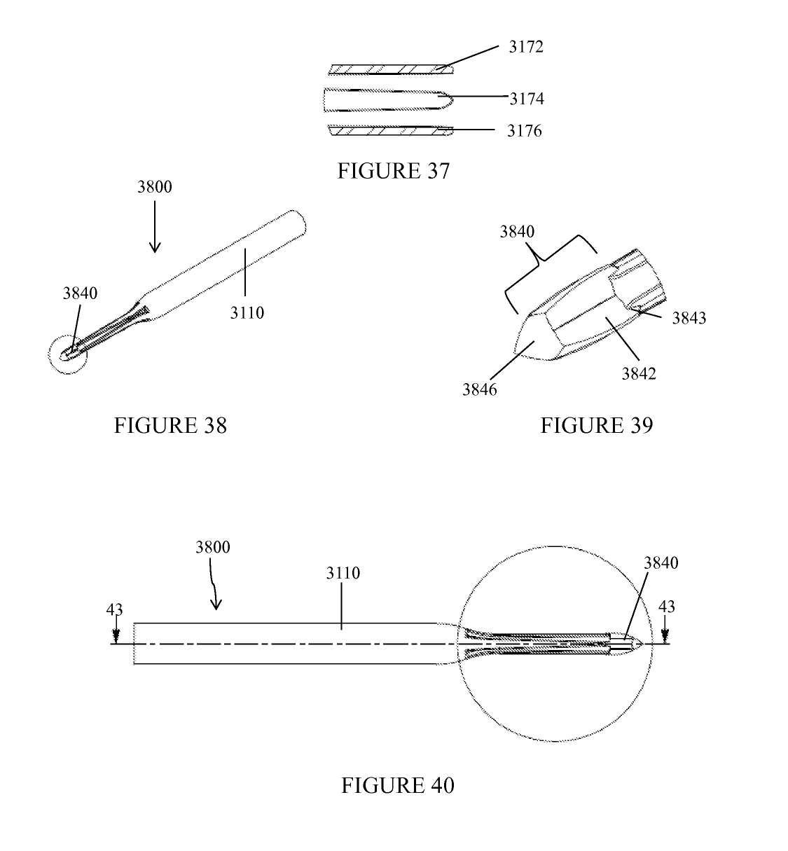

In some embodiments, the second section 130 may be configured to be partially and/or entirely inserted within a treatment site. The second section 130 may have varied shapes and configurations. The different shapes and configurations are described with respect to FIGS. 1-37.

In some embodiments, the second section 130 may have a uniform diameter. In other embodiments, the second section 130 may have a tapered diameter. In some embodiments, the second section 130 may have a diameter that gradually gets smaller along its length toward the delivery opening 132 to reduce the stress asserted on the agents when being delivered through the delivery device. In some embodiments, the second section 130 may include a plurality of portions.

In some embodiments, as shown in FIGS. 1 through 3, the second section 130 may include a first portion 134 and a second portion 136. In some embodiments, one, some, or all of the dimensions of the first portion 134 and the second portion 136 may be the same. In other embodiments, one, some, or all of the dimensions of the first portion 134 and the second portion 136 may be different. In some embodiments, for example, the first portion 134 and the second portion 136 may differ in length, diameter, shape, or a combination thereof.

In some embodiments, as shown in FIGS. 1 through 3 and 9 through 12, the second portion 136 may have a smaller diameter than the first section 120. The first portion 134 may have a tapered diameter in which the diameter gradually decreases along the length towards the delivery opening 132 from about the diameter of the first section 120 to the diameter of the second portion 136.

In some embodiments, the delivery body 110 may include a chamfered surface 138 that surrounds the delivery opening 132. The chamfered surface 138 may be limited to the outer surface of the delivery body 110 and thereby not affect the diameter of the inner channel 102.

In some embodiments, the diameter of the inner channel 102 may correspond to the diameters of the first and second sections 120 and 130. For example, as shown in FIG. 12, the diameter of the inner channel 102 may be planar in the first section 120 and the second portion 136, and the diameter of the inner channel 102 may be tapered in the first portion 134.

In some embodiments, the delivery body 110 may include a coating on the outer surface to aid the placement of the delivery body 110. For example, the outer surface may be treated with active agents to prevent platelet formation and blood clotting, such as an anti-coagulant coating; a lubricant, such as a water-based, water-soluble lubricant; other materials, such as radiopaque coating material; or a combination thereof.

In some embodiments, the retractable member 140 may include a first (proximal) end 142, an opposing second (distal end) 144, and a length therebetween. In some embodiments, as shown in FIGS. 1 through 3, the retractable member 140 may be concentric partially or entirely along its length. In other embodiments, the retractable member 140 may have a different shape.

In some embodiments, the retractable member 140 may have a diameter along at least a portion of its length configured to seal the delivery opening 102 to prevent leakage while being inserted at the treatment site. In some embodiments, the retractable member 140 may have the same diameter along its length. In other embodiments, the retractable member 140 may have a diameter that varies along its length.

In some embodiments, the retractable member 140 may be completely solid as shown in FIGS. 1 through 3. In other embodiments, the retractable member 140 may be partially solid, or a combination thereof. In other embodiments, the retractable member 140 may be partially or completely hollow.

In some embodiments, the retractable member 140 may be rigid in at least one direction. In some embodiments, the retractable member 140 may be radially rigid. In other embodiments, the retractable member 140 may be entirely or partially flexible in at least one direction. In some embodiments, a portion of the retractable member 140 along its length may be flexible.

In some embodiments, the retractable member 140 may be configured to create at least one delivery channel at the treatment site by cutting or piercing the tissue to separate the tissue at the treatment site. In some embodiments, the retractable member 140 may include at least one puncture member 146 configured to penetrate the treatment site to form a delivery channel for the agent to be delivered by the delivery device. In some embodiments, the puncture member 146 may be configured to separate tissue rather than core the tissue at the treatment site. In other embodiments, the puncture member 146 may be configured to partially or entirely core tissue at the treatment site.

In some embodiments, the retractable member 140 may include one puncture member 146 disposed at the second end 144. In other embodiments, the retractable member 140 may include additional puncture member(s) disposed along its length. For example, the retractable member 140 may include one, two, three, or more than three puncture members along its length. The puncture members may have the same or different shape and/or configuration.

In some embodiments, the puncture member 146 may include a sharp tip. In some embodiments, the puncture member 146 may include at least one bevel surface. As shown in FIGS. 1 through 3, the puncture member 146 may include one beveled surface 148 that transverses the diameter. In other embodiments, the puncture member 146 may include more than one bevel surface.

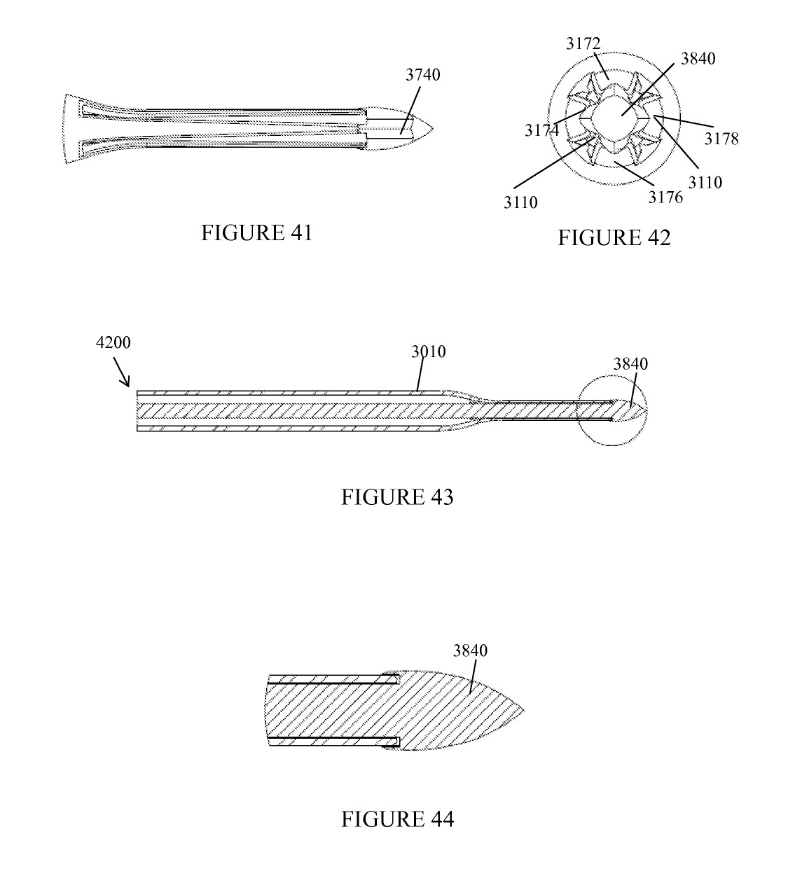

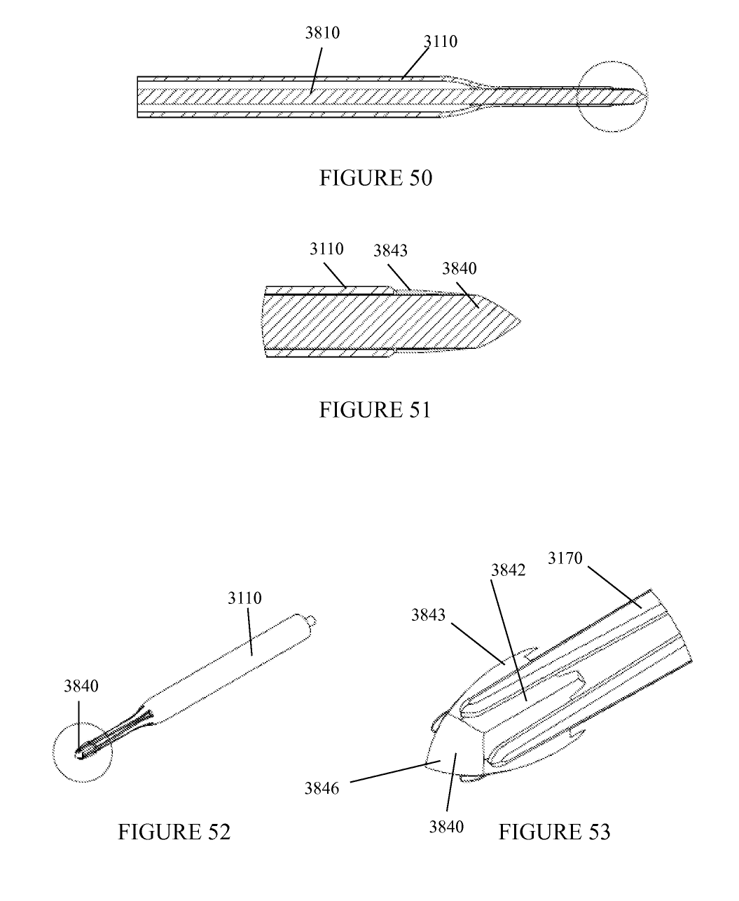

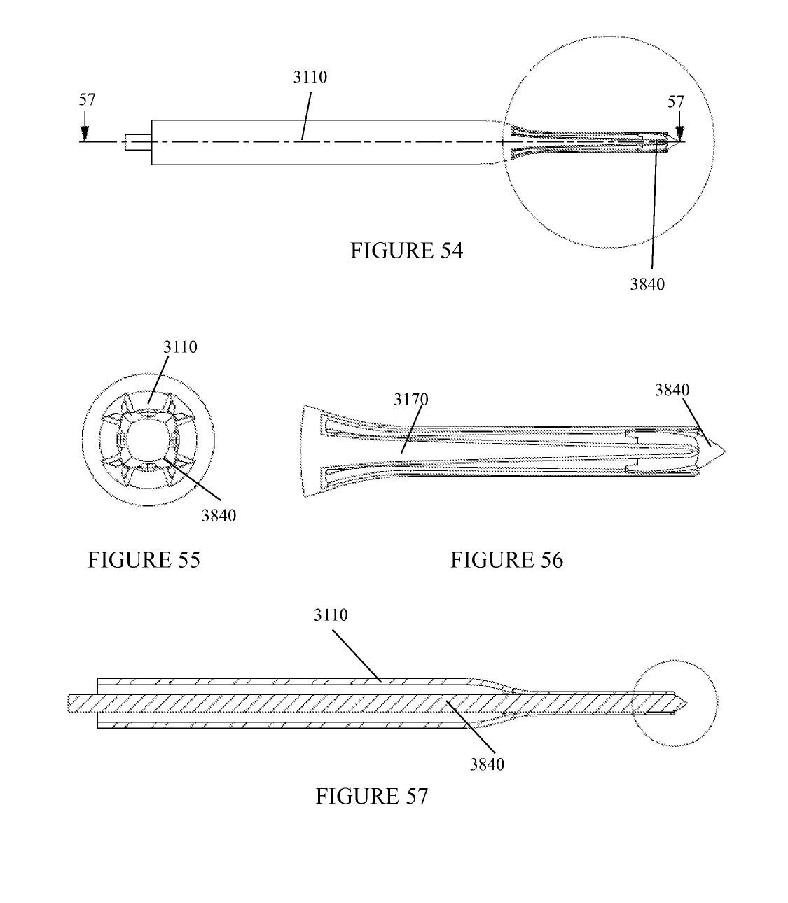

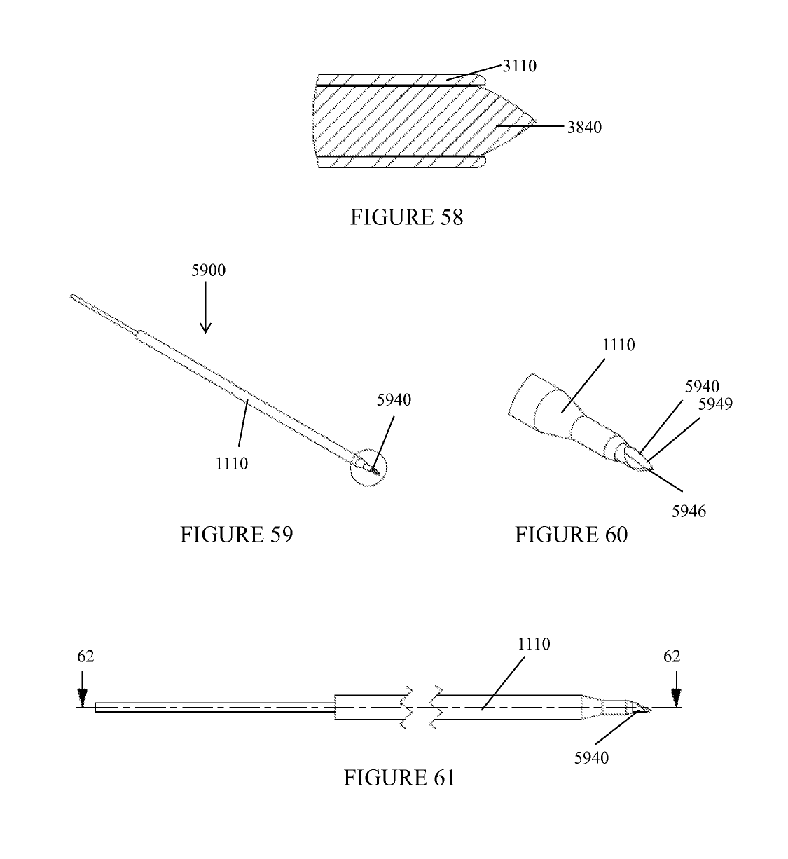

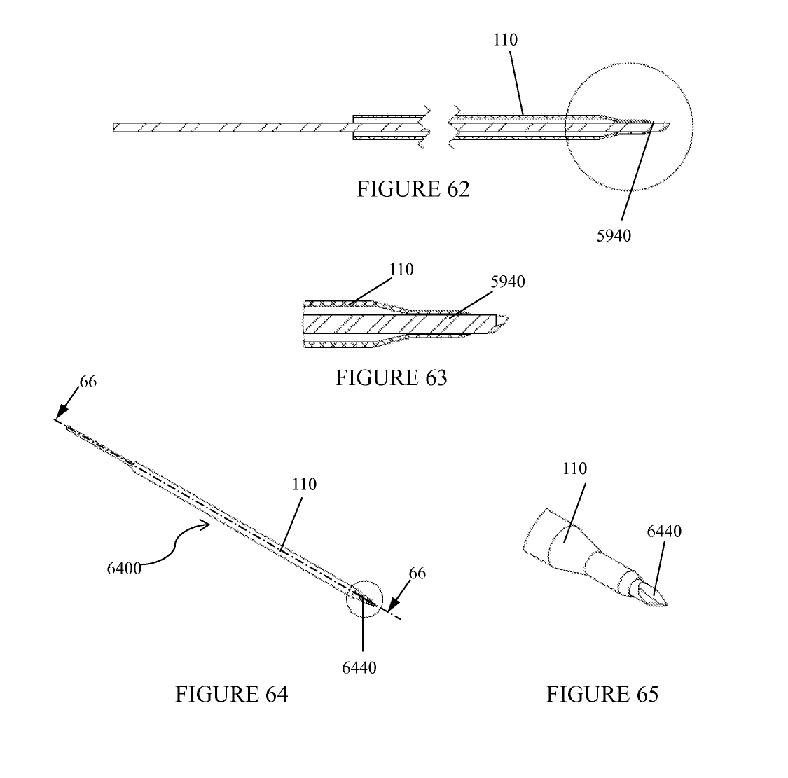

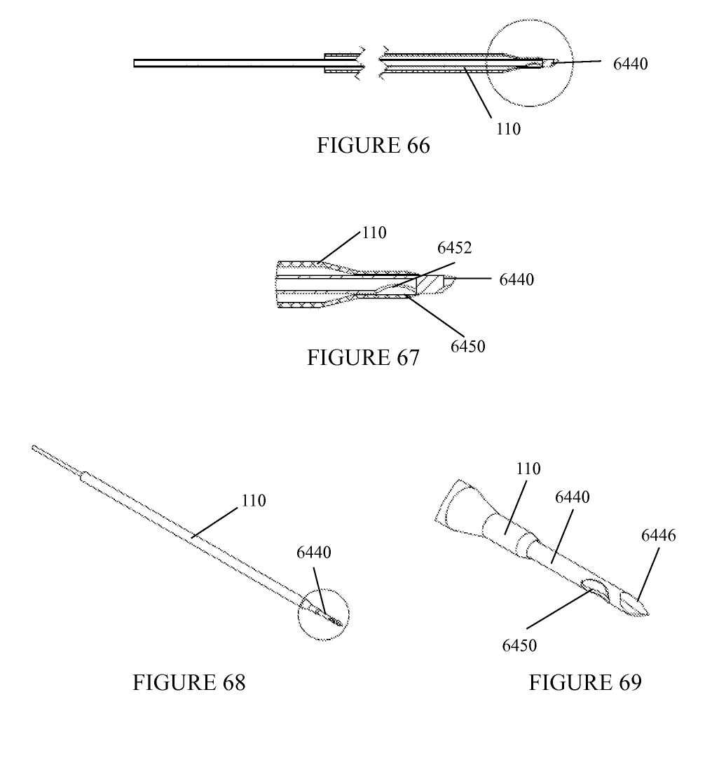

In some embodiments, the puncture member 146 may have a single faceted, multi-faceted shape, conical shape, or a combination thereof. In other embodiments, the puncture member 146 may have a different shape. FIGS. 1-8, 14-16, 23-26, and 38-80 show additional embodiments of the retractable member and/or the puncture member. The shape of the puncture member may depend on any one of the delivery body, tissue type, location, and access path of the treatment site.

In some embodiments, the sharpness of the puncture member 146 may depend on the tissue of the treatment site. In some embodiments, a portion of the puncture member 146 may be blunt. In some embodiments, the puncture member 146 may include a cutting surface or edge configured cut or pierce to create an incision upon tissue penetration of the treatment site.

The retractable member 140 may be configured to be movable with respect to the delivery body 110. The retractable member 140 may be movable within the inner channel of the delivery body. In some embodiments, the retractable member 140 may move between a resting or closed position (a first position), an extended position (a second position), and a retracted position (a third position).

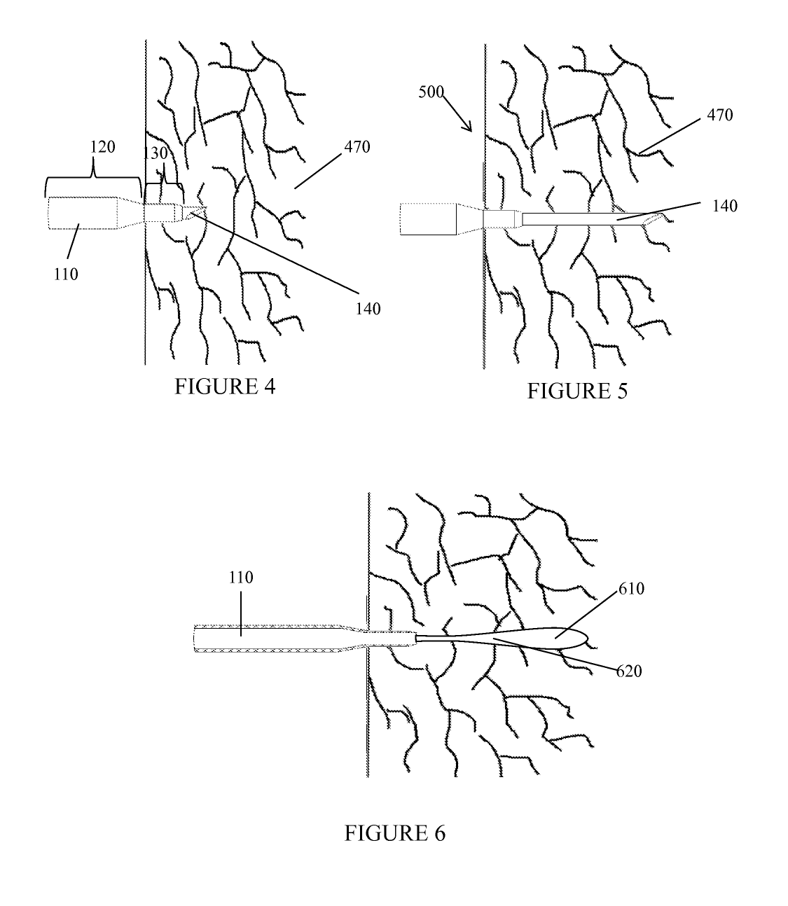

In some embodiments, the resting position may include when the retractable member 140 does not substantially extend past or is not substantially external to the second end 114 of the delivery body 110. The resting position may include when a portion of the retractable member 140 that includes the puncture member 146 extends past the second end 114. In the resting position, the retractable member 140 may be positioned so that a first portion 141 of the retractable member 140 is disposed outside of the delivery body adjacent to the first end 112 of the delivery body 110, a second portion 143 of the retractable member 140 disposed within the delivery body 110 between the first end 112 and the second end 114, and a third portion 145 of the retractable member 140 is disposed outside of the delivery body adjacent to the second end 114 of the delivery body 110. In some embodiments, the third portion 145 may be longer than the first portion 141. The third portion 145 may include the puncture tip 146. FIGS. 1 and 4 show the retractable member 140 positioned with respect to the delivery body 110 at the resting position.

In some embodiments, the extended position may include when the portion of the retractable member 140 that extends past the second end is increased. The extended position may include when the retractable member 140 is extended into a treatment site. In the extended position, the retractable member 140 may be positioned so that the third portion 145 of the retractable member 140 has increased in length and the first portion 141 has decreased in length as compared to the resting position. In the extended position, the retractable member 140 may be configured to penetrate the treatment site to form a delivery channel for the agent to be delivered by the delivery device. FIG. 3 shows the retractable member 140 positioned with respect to the delivery body in the extended position (the second position).

In some embodiments, the retracted position may include when the retractable member 140 is retracted within the delivery body 130 towards the proximal end 112 so that the retractable member 140 is either partially or entirely removed from the delivery body 110. When the retractable member 140 is in the retracted position, the delivery body 110 may be configured so that an agent may be delivered through the opening 132 of the delivery body 110.

In some embodiments, the delivery device may be attached or connected to a delivery system. As shown in FIGS. 1 and 3, the delivery device 100 may be attached to delivery system 160. The delivery system 160 may include a handle assembly. The delivery system is not limited to a handle system and may be any known delivery system. The delivery device 100 may include an adapter 150 disposed at the proximal end 112 of the delivery body. The delivery system 160 may be configured to control the position of the retractable member 140, to control and position the delivery body 100, and/or may be configured to control the delivery of an agent.

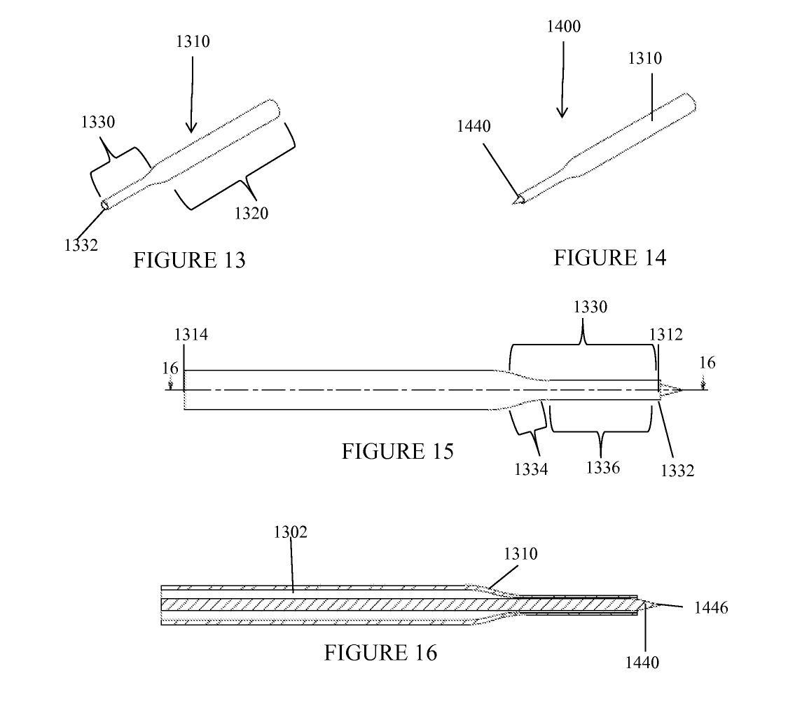

FIGS. 4 through 8 illustrate an example of operation of a delivery device according to embodiments. The delivery device shown corresponds to the embodiments to the delivery device shown in FIGS. 1 through 3 and 9 through 12. However, it is understood that the operation of the delivery device shown in FIGS. 4-8 also describes the operation of a delivery device according to the other embodiments described in FIGS. 13 through 83.

In operation, a clinician positions the delivery device 100 with respect to a treatment site 470. In some embodiments, the delivery device 100 is positioned with the retractable member 140 leading into the tissue. The delivery device 100 may be positioned so that the second section 130 of the delivery body 110 along with the puncture member 146 of the retractable member 140 is disposed within the treatment site and the first section 120 of the delivery body 110 is disposed adjacent to the treatment site 470, as shown in FIG. 4.

Next, as shown in FIG. 5, the clinician may cause the retractable member 140 to extend into the treatment site 470 to form the delivery channel 610. After the delivery channel 610 is formed at the treatment site, the retractable member 140 may be retracted thereby forming a delivery channel 610 within the treatment site 470. As shown in FIG. 6, the retractable member 140 may partially or completed retracted from within the delivery body 110. In some embodiments, the clinician may cause the delivery of a solution 620 to dilate the delivery channel 610 before the agents are delivered. The solution 620 may be any biocompatible solution and may include but is not limited to saline.

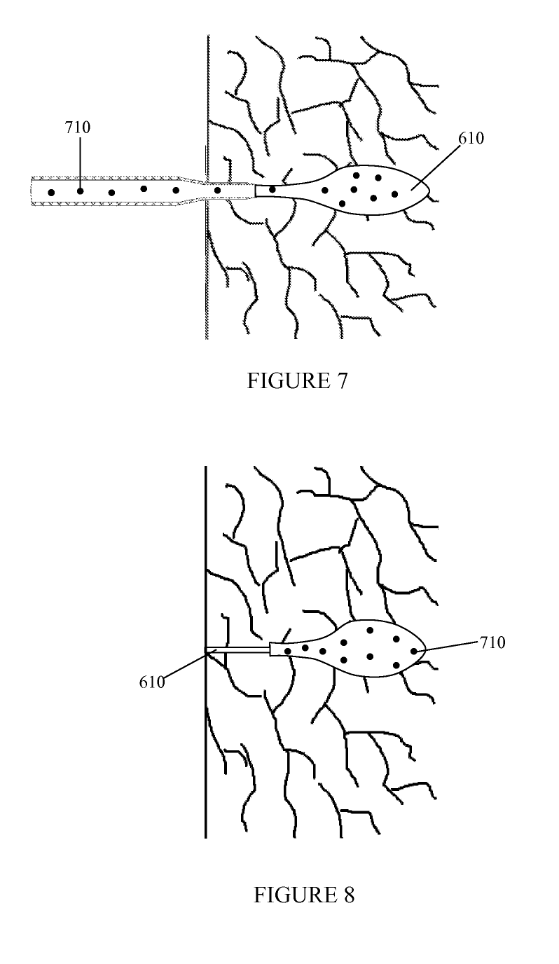

Next as shown in FIG. 7, the clinician may cause a therapeutic agent 710 to be delivered. Because the delivery body has a wider bore, the stress on the agent may be reduced as compared to delivering the agent through needles. The therapeutic agent may be delivered into the channel. The lead agents may cause the channel to dilate in advance of the other agents. After the agent is delivered, the delivery body 110 may be retracted. As shown in the figures, the delivery body may be configured to remain in the same position in the treatment site from initial positioning through agent delivery. As shown in FIG. 8, the therapeutic agent 710 may remain within the treatment site 470 after the delivery device 100 is removed.

In some embodiments, the retractable member 140 and the delivery body 110 may be made of the same or different biocompatible materials. In some embodiments, the retractable member 140 may be made of material having sufficient strength to withhold most of the stress associated with advancing the delivery body 110 to the treatment site.

In some embodiments, the retractable member 140 and/or the delivery body 110 may be made of a stiff or rigid material. In other embodiments, the retractable member 140 and/or the delivery body 110 may be made partially or entirely of a flexible material or a semi-flexible. In some embodiments, the delivery body 110 and/or the retractable member 140 may have different regions of flexibility. In some embodiments, the delivery body 110 and/or the retractable member 140 may be made of one or more polymeric materials that may be in combination with a metallic reinforcement.

The materials may include but are not limited to one or more the following materials: metallic alloys, shape memory or pseudoelastic alloy (e.g., Nitinol), thermoplastic elastomer, urethane, polymer, polypropylene, plastic, ethylene chlorotrifluoroethylene (ECTFE), polyterafluoroethylene (PFFE), fluorinated ethylene propylene copolymer (FEP), nylon, polyurethane, silicone, combinations thereof, and the like.

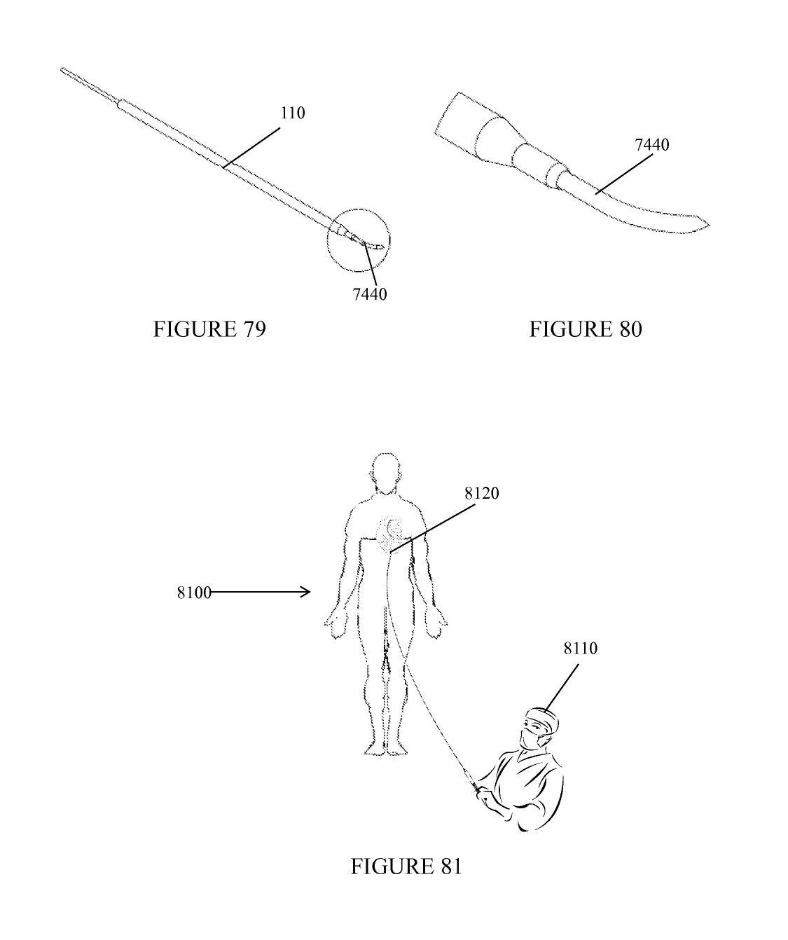

FIGS. 13 through 83 show examples of other embodiments of the delivery device shown in FIGS. 1 through 12. The delivery device shown in FIGS. 1 through 12 may be modified to include a delivery body and/or a retractable member according to any one of these embodiments. It would be understood that the embodiments of the delivery body, retractable member, and delivery device described with respect to FIGS. 1 through 12 may also apply to the delivery bodies, retractable members, and delivery devices described with respect to FIGS. 13 through 83. The delivery bodies, retractable members, and delivery devices described with respect to FIGS. 13 through 83 may be similar with some respects to the delivery body, retractable member, and delivery device described with respect to FIGS. 1 through 12. It will also be understood that the configurations of retractable members and delivery bodies shown in FIGS. 13 through 79 are not limited to those shown in the figures and may include other configurations.

### Delivery Body

FIGS. 13 through 24 and 31 through 37 show examples of delivery devices that include delivery bodies, as well as delivery bodies according to other embodiments. It will be understood that the delivery body according to embodiments may include any combination of the embodiments shown in these figures, as well as FIGS. 1 through 12.

In some embodiments, the delivery body may be configured to provide additional support to the created delivery channel and/or to dilate the created delivery channel. In some embodiments, the delivery body may be configured so that a longer length of the delivery body may be inserted into and retained in the treatment site during the delivery of a therapeutic agent.

In some embodiments, the second section of the delivery body may be configured to be further inserted into the treatment site. In some embodiments, the second portion of the second section may have a length second portion that is longer than a length of the first portion. FIGS. 13 through 16 show a delivery body 1310 according to these embodiments.

As shown in FIGS. 13 through 16, the delivery body 1310 may include a first end 1312, an opposing second end 1314, and a length therebetween. In some embodiments, the delivery body 1310 may include a first section 1320 and a second section 1330. The first section 1320 may be adjacent to a first end 1312 and the second section 1330 may be adjacent to a second end 1314. The delivery body 1310 may include an inner channel 1302 that extends along its length from the first end 1312 to the second end 1314. The delivery body 1310 may also include a delivery opening 1332 disposed at the second end 1314.

The second section 1330 may include a first portion 1332 and a second portion 1334. A length of the second portion 1334 may be longer than a length of the first portion 1332. The second portion 1334 may have a smaller diameter than the first second 1320 and the first portion 1332 may have a tapered diameter in which the diameter gradually decreases along the length towards the delivery opening 1332 from about the diameter of the first section 1320 to the diameter of the second portion 1336.

In some embodiments, the delivery body may include a plurality of ports along at least a portion of its length. The ports may be configured to deliver and disperse a therapeutic agent within the created delivery channel. The ports may be configured to locally deliver and diffuse a therapeutic agent within a portion of the created delivery channel that surrounds a portion of the delivery body. The ports may be disposed along delivery body so that the therapeutic agents may be diffused directly from the inner channel and thereby reducing the shear stress caused by traveling through a smaller lumen.

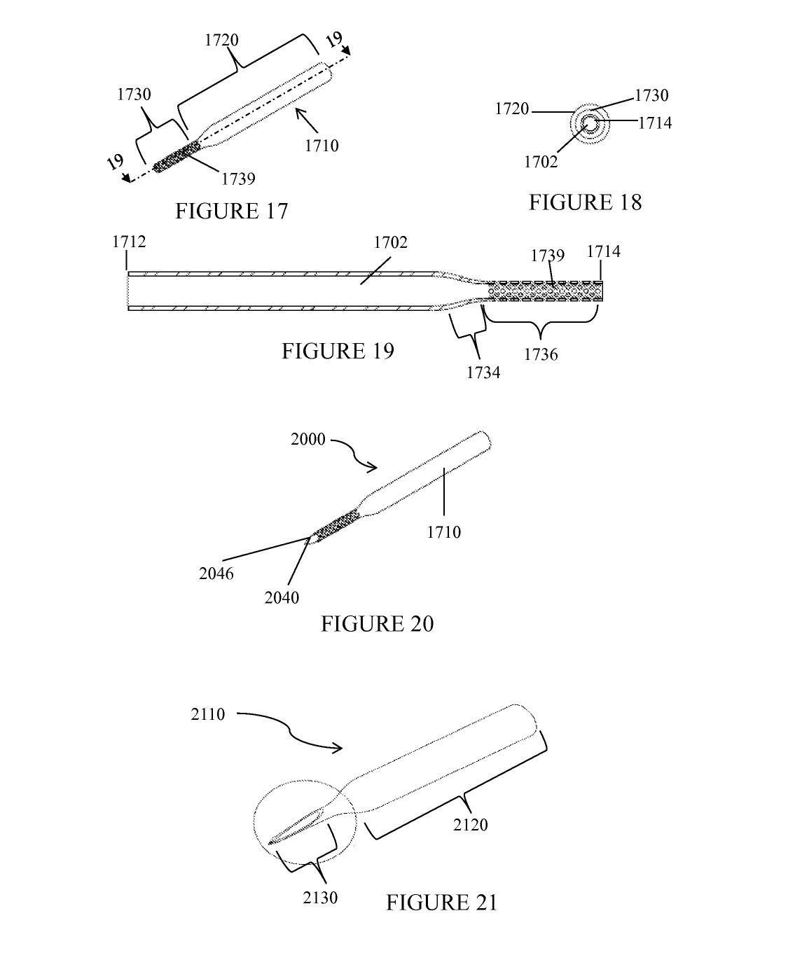

FIGS. 17 through 20 show an example of a delivery body 1710 that includes a plurality of ports 1739. However, it will be understood that a delivery body according to different embodiments may include ports.

The delivery body 1710 may be similar to the delivery body 1310. Like the delivery body 1310, the delivery body 1710 may include a first section 1720 and a second section 1730. In some embodiments, the delivery body 1710 may include a first section 1720 and a second section 1730. The first section 1720 may be adjacent to a first end 1712 and the second section 1730 may be adjacent to a second end 1714. The delivery body 1710 may include an inner channel 1702 that extends along its length from the first end 1712 to an opposing second end 1714. The delivery body 1710 may also include a delivery opening 1732 disposed at the second end 1714

The delivery body 1710 may include a plurality of ports 1739 disposed along its length. In some embodiments, the ports 1739 may be disposed partially and/or entirely along the length of the second section 1730. In some embodiments, the ports 1739 may be disposed along a portion of the second section 1730 of the delivery body. In some embodiments, the ports 1739 may be disposed along the entire second portion of the second section 1730.

The ports 1739 may be of any size, number, and/or configuration. The ports 1739 may be of the same size, different sizes, or a combination thereof. In some embodiments, the ports 1739 may have a circular shape, for example, as shown in FIGS. 17 through 20. In other embodiments, the ports 1739 may have a different shape. The delivery body 1710 may include any number of ports 1739.

In some embodiments, the ports 1739 may be evenly spaced along the length of the delivery body. In other embodiments, the ports 1739 may be staggered along the length of the delivery body, for example, as shown in FIGS. 17 through 20.

The delivery body may include an inner surface and an outer surface. In some embodiments, the inner surface and the outer surface of the second section of the delivery body may be a concentric continuous surface, for example, as shown in FIGS. 1 through 20. In some embodiments, the delivery body may include a delivery opening that is substantially planar and/or perpendicular to the length of the delivery body, as shown in FIGS. 1 through 20. In other embodiments, a portion of the surfaces of the delivery body may be discontinuous.

In some embodiments, the delivery opening may be transverse with respect to the length of the delivery body. In some embodiments, a portion of the surfaces of the delivery body may extend beyond the other portion of the surfaces of the delivery body so that the inner surface may be exposed. The extended surface(s) may be configured to hold the delivery channel open so that when the agent is delivered through the delivery opening, the back pressure and the resistance to the delivery of the agents may be reduced. FIGS. 21 through 30 show examples of a delivery body according to these embodiments.

In some embodiments, the delivery body may include at least one puncture member configured to puncture and create the delivery channel and disposed near the delivery opening. In some embodiments, the puncture member may include a puncture tip. In some embodiments, the puncture member may additionally or alternatively include at least one cutting surface or edge disposed along at least a portion of the surface of the delivery body that surrounds the delivery opening.

The sharpness of the cutting edge and/or punctured tip may be the same or different. The sharpness may vary and depend on the location of the treatment site. In some embodiments, the cutting edge may be configured to create an incision upon exposure and penetration. In some embodiments, the cutting edge may be configured to blunt so as to avoid coring or excessive damage to the treatment site when the delivery body

FIGS. 21 through 24 show a delivery body 2110 with a delivery opening 2132 having a puncture tip and/or cutting edge. Like the delivery body 1710, the delivery body 2110 may include a first section 2120 and a second section 2130 that includes a first portion 2134 and a second portion 2136. The second section may 2130 may have a smaller diameter than the first section 2120, wherein the first portion 2134 may have a tapered diameter and the second portion 2136 may have a diameter smaller than the first section 2120. The delivery body 2110 may have an internal channel 2102 along the length between a first end 2112 and a second end 2114. A delivery opening 2132 may be disposed at the second end 2114.

As shown in FIGS. 21 through 24, the delivery opening 2132 may be disposed so as to be transverse to the length of the delivery body 2110. The delivery opening may traverse the length so that one side of the second section extends longer than the other side so that an inner surface 2111 of the delivery body 2110 may be exposed. As shown in the enlarged view, a portion of the delivery opening 2132 may include a puncture member 2131. In some embodiments, the delivery opening 2132 may include at least one beveled surface 2133, wherein the opening is disposed adjacent thereto or therethrough. The beveled surface 2133 may be configured to be a cutting edge. In some embodiments, the delivery opening 2132 may include a punctured tip 2131 disposed at the end of the exposed inner surface 2111.

In some embodiments, the delivery body may include a plurality of extending members that are configured to reduce the shear resistance by supporting the delivery channel for delivery of the agent. In some embodiments, the second section of the delivery body may include a plurality of extending members that surround the delivery opening. FIGS. 31 through 37 show a delivery body 3110 with a plurality of extending members 3170.

The delivery body 3110 has a similar structure as delivery body 2110 but for the second section 3130. Like the delivery body 2110, the delivery body 3110 may include a first section 3120 and a second section 3130 that includes a first portion 3134 and a second portion 3136. The second section may 3130 may have a smaller diameter than the first section 3120, wherein the first portion 3134 may have a tapered diameter and the second portion 3136 may have a diameter smaller than the first section 3120. The delivery body 3110 may have an internal channel 3102 along the length between a first end 3112 and a second end 3014. The delivery body may include a plurality of extending members 3170 that extend from the length and that surround a delivery opening 3132.

There may be any number of the extending members 3170. In some embodiments, the protruding members may include four extending members 3172, 3174, 3176, and 3178, as shown in FIGS. 31 through 37. In other embodiments, there may be more or less extending members 3170, for example, two, three, five, six, seven, eight, or more than eight.

The extending members 3170 may have a same shape, a different shape, or a combination thereof. In some embodiments, the extending member(s) 3170 may have a tapered shape. The width of the extending members 3170 may decrease along the length of an extending member. As shown in FIG. 34, the width may decrease along the length of the extending member 3174 from the second portion towards the second end 3114. In other embodiments, the extending member(s) 3170 may have the same width along its length.

In some embodiments, the extending members 3170 may be evenly spaced around the delivery opening 3132. In other embodiments, the extending members 3170 may be unevenly spaced.

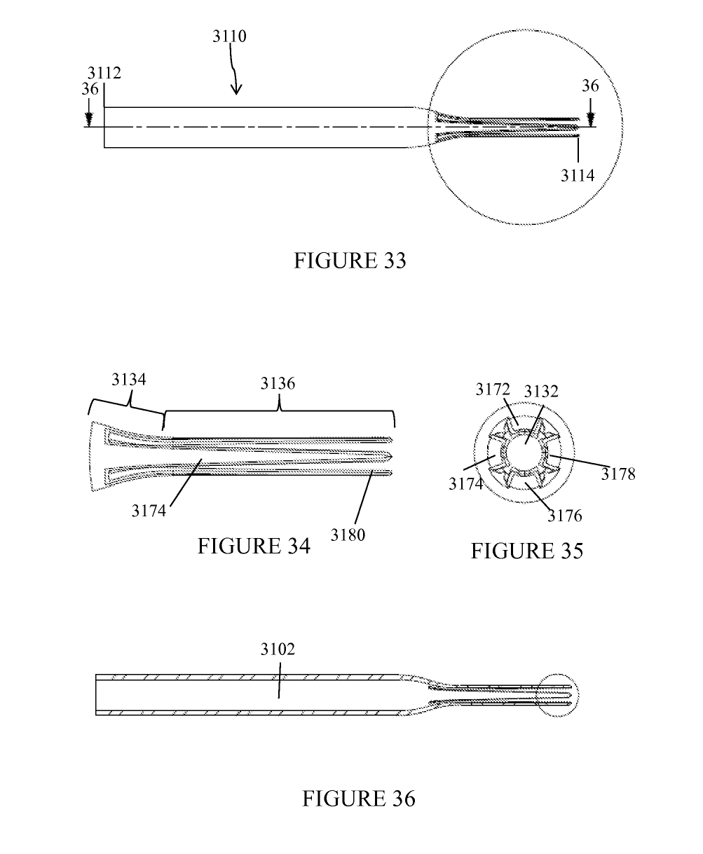

In some embodiments, the delivery body 3110 may include spaces 3180 between each of the extending members 3170. The width of the spaces 3180 may correspond to the width of the extending members 3170. For example, if the width of the extending members 3170 is tapered, the width of the spaces may be reversely tapered. As shown in FIG. 33, the width of the space 3180 gradually increases as the width of the extending member 3174 gradually decreases.

In some embodiments, the ends 3171, 3173, 3175, and 3177 of the respective extending members 3172, 3174, 3176, and 3178 may have any shape. In some embodiments, the ends 3171, 3173, 3175, and 3177 may have a same shape, different, shape, or a combination thereof. In some embodiments, an end 3171, 3173, 3175, and/or 3177 may have a rounded or blunt shape. In other embodiments, an end 3171, 3173, 3175, and/or 3177 may have a beveled shape. In some embodiments, an end may have a pointed shape. In some embodiments, an end 3171, 3173, 3175, and/or 3177 may be configured to create an incision upon exposure and penetration.

In some embodiments, the extending members 3170 may extend from a portion of the second section 3130. In some embodiments, the extending members 3170 may extend from the first portion 3120. In other embodiments, the extending members 3170 may extend from another portion along the length of the delivery body 3110.

In some embodiments, the extending members 3170 may be flexible or partially flexible. In some embodiments, the extending members 3170 may be configured to partially expand when released and contract when retained by a retractable member. In some embodiments, the extending members 3170 may be configured to expand or open to support and/or expand the delivery channel when delivering a therapeutic agent.

In some embodiments, the delivery body may be used with a retractable member according to embodiments, for example, as shown in FIGS. 38-58. In other embodiments, the delivery body may be used with a different retractable member.

### Retractable Member

FIGS. 14 through 16, 20, 25 through 30, and 39 through 78 show examples of delivery devices that include retractable members according to other embodiments. It will be understood that the retractable member according to embodiments may include any combination of the embodiments shown in these figures, as well as FIGS. 1 through 12.

In some embodiments, the retractable member may be configured to occlude one, some or all of the openings when the delivery device is being advanced to the treatment site. In some embodiments, the body of the retractable member may be configured to seal the opening(s) disposed on the delivery body. In other embodiments, an end of the retractable member may be configured to partially or completely seal the delivery opening.

In some embodiments, the retractable member may be configured to move radially and/or axially with respect to the delivery body.

In some embodiments, the retractable member may include a puncture member disposed at one end. In some embodiments, the puncture member may be configured to create an incision upon tissue penetration of the treatment site. In some embodiments, the puncture member may be configured to separate the tissue rather than core the tissue. In other embodiments, the puncture member may be configured to core the tissue. The shape and configuration surfaces of the puncture member may vary depending on the desired sharpness of the puncture member. The shape of the puncture member may include but is not limited to a single faceted, multi-faceted shape, conical shape, or a combination thereof. The sharpness may vary and depend on the location of the treatment site.

In some embodiments, the puncture member may have a conical shape. FIGS. 14 through 16 and 20 show examples of a conical puncture member.

In some embodiment, the puncture member may be sharp. As included in the delivery device 1400 shown in FIGS. 14 through 16, a retractable member 1440 may include a puncture member 1446 with a conical shape. As shown in these figures, the puncture member 1446 may include a tip wherein the surfaces of the retractable member 1440 converge. In some embodiments, the tip and surfaces may be sharp.

In other embodiments, the puncture member may have a conical shape that is smooth and/or blunt. As included in the delivery device 2000 shown in FIG. 20, a retractable member 2040 may include a puncture member 2040 with a conical shape. As shown in these figures, the puncture member 2046 may include a tip wherein the surfaces of the retractable member 2040 converge. In some embodiments, the surfaces and/or tip may be curved and/or rounded.

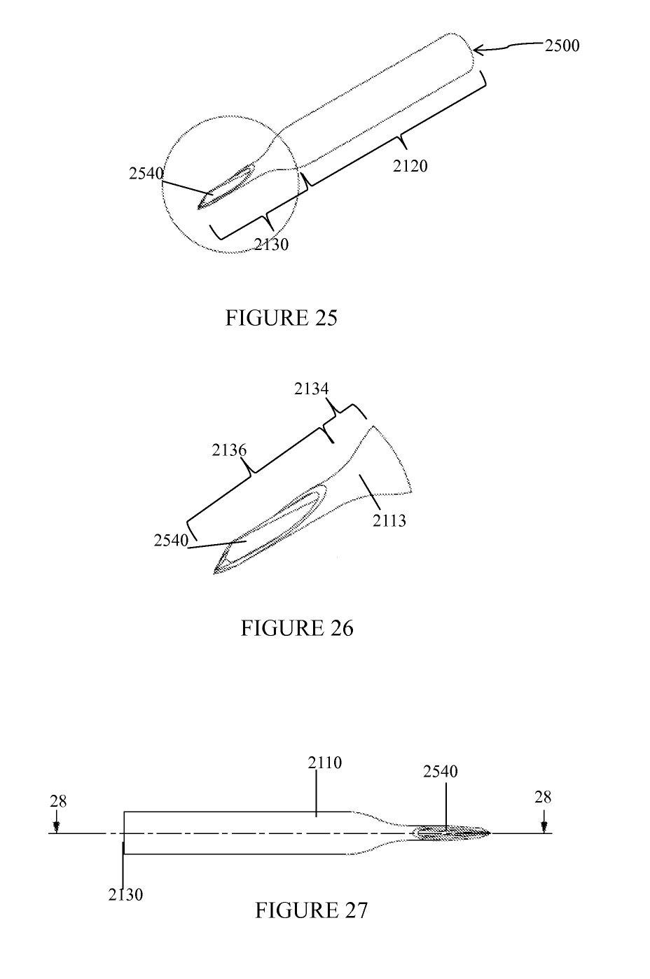

In some embodiments, the puncture member may include at least one multifaceted beveled edge and/or surfaces. FIGS. 25 through 30 show example of a delivery device 2500 with a retractable member 2540 having a multifaceted puncture member 2546. The puncture member 2546 may include a beveled edge 2547 transverses the diameter and at least one beveled edge 2549 that traverses the length along one side of the surfaces. In some embodiments, the beveled edge(s) 2549 may be configured to converge at a sharp tip. In some embodiments, the delivery opening 2132 of the delivery body 2110 may be configured to expose portions of the retractable member 2549 while the delivery device 2500 is being advanced to the treatment site.

FIGS. 59 through 63 show another example of a delivery device 5900 with a retractable member 5940 having a multi-faceted puncture member 5946. The puncture member 5946 may include a plurality of beveled edges 5949 that traverse to both the diameter and length of the retractable member 5940. In some embodiments, the beveled edge(s) 5949 may be configured to converge at a sharp or cutting edge.

In some embodiments, the retractable member may have a shape that complements the shape of the delivery body and/or delivery opening. In some embodiments, the puncture member may include a plurality of protruding members configured to mate with the delivery body. In some embodiments, the protruding members may compliment the extending members of the delivery body so as to move with respect to the delivery body in a plurality of positions. In some embodiments, the protruding members may be configured to retain the extending members in a closed position when the delivery device is being advanced to the treatment site. In some embodiments, the retractable member may be configured to be spring loaded with respect to the delivery body when in a closed position. The retractable member may be configured to rotate with respect to the delivery body to an open or delivery position and/or retractable position. The retractable member may be configured to rotate to release the extending members of the delivery body. In some embodiments, the retractable member may be made of a pseudo-elastic material, such as a memory shape alloy. FIGS. 38 through 58 show examples of a retractable member according to these embodiments.

FIGS. 38 through 44 show a delivery device 3800 including a retractable member 3840 and the delivery body 3110 according to some embodiments in a closed position. The retractable member 3840 may include a plurality of protruding members 3843 protruding from a puncture member 3846. The protruding members 3843 may extend in the direction opposite of the extending members 3170. In some embodiments, the retractable member may include at least one curved surface 3842 between each protruding member. The curved surface 3842 may be configured to move under the extending members 3170 when the retractable member is rotated to be retracted. In some embodiments, the retractable member 3840 may include a beveled surface transverse to the diameter that converges at a tip.

As shown in FIGS. 38 through 44, the protruding members 3843 may be configured to extend over and retain the plurality of extending members 3172, 3174, 3176, and 3178 of the delivery device 3110 so that the delivery device is in a closed position.

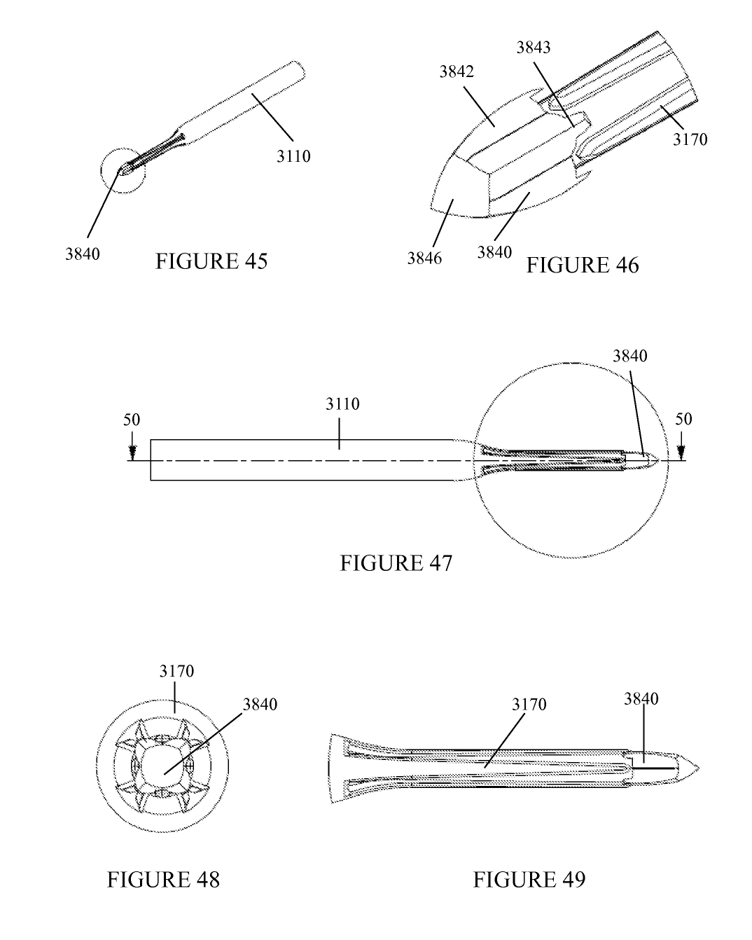

FIGS. 45 through 51 show the delivery device 3800, wherein the retractable member 3840 is in an open or extended position with respect to the delivery body 3110. In this position, the delivery device 3800 may be configured to deliver a therapeutic agent via the spaces 3180 between each of the extending members 3170. The retractable member 3840 may be configured to rotate with respect to the delivery body 3110 so that the protruding members 3843 no longer cover and retain the extending members 3170. In the open position, the extending members may be released from the tension caused by the protruding members 3843 and may expand.

FIGS. 52 through 58 show the delivery device 3800, wherein the retractable member 3840 is in a partially retracted position with respect to the delivery body 3110. After the retractable member 3840 has been released by rotating the retractable member with respect to the delivery body 3110, the retractable member 3840 may be retracted. As shown in FIG. 53, the retractable member 3840 may be configured to be retracted from the delivery body 3810 by aligning the curved surfaces 3842 with each of the extending members 3170 so that the extending members 3170 pass over the curved surfaces 3842 when the retractable member 3840 is retracted.

In some embodiments, the retractable member may be configured to expand the formed delivery channel. In other embodiments, the retractable member may include one puncture member disposed at an end and configured to form a delivery channel. In other embodiments, the retractable member may include more than one puncture member. The additional puncture member(s) may be configured to expand the delivery channel.

In some embodiments, the retractable member may include at least one (a second) puncture member disposed along its length. The puncture member may be configured to core and remove tissue from the delivery channel at the treatment site. The puncture member may be configured to expand the delivery channel from by the other (first) puncture member. In some embodiments, the puncture member may be configured to increase the diameter of the delivery channel by rotating and removing tissue surrounding the delivery device.

FIGS. 64 through 69 show an example of a delivery device 6400 with a retractable member 6440. The retractable member 6440 may include at least a (first) puncture member 6446 disposed at an end and a (second) puncture member 6450 disposed along the length. The puncture member 6446 may be like puncture member 5940 or may be according to different embodiments. The retractable member may include a plurality of (second) puncture members spaced along its length and/or circumference.

The second puncture member 6450 may be disposed along the length of the retractable member 6440. The second puncture member 6450 may be disposed adjacent to the first puncture member 6446. In some embodiments, the second puncture member 6450 may include a tissue removal port 6452 configured to retain the removed tissue. The tissue removal port 6452 may be a depression. In some embodiments, the tissue removal port 6452 may communicate with a suction source via an internal lumen or channel provided within the retractable member 6440.

In some embodiments, the puncture member 6450 may have a cutting edge partially or entirely surrounding the tissue removal port configured to penetrate and incise the tissue surrounding the delivery channel and/or device. The depression may be configured to retain the tissue incised by the cutting edge for removal, thereby expanding the delivery channel formed by the (first) puncture member of the retractable member.

In some embodiments, the retractable member may be solid. In some embodiments, portions of the retractable member may be solid. In some embodiments, the retractable member may be partially hollow. In some embodiments, the retractable member may include a channel along its length.

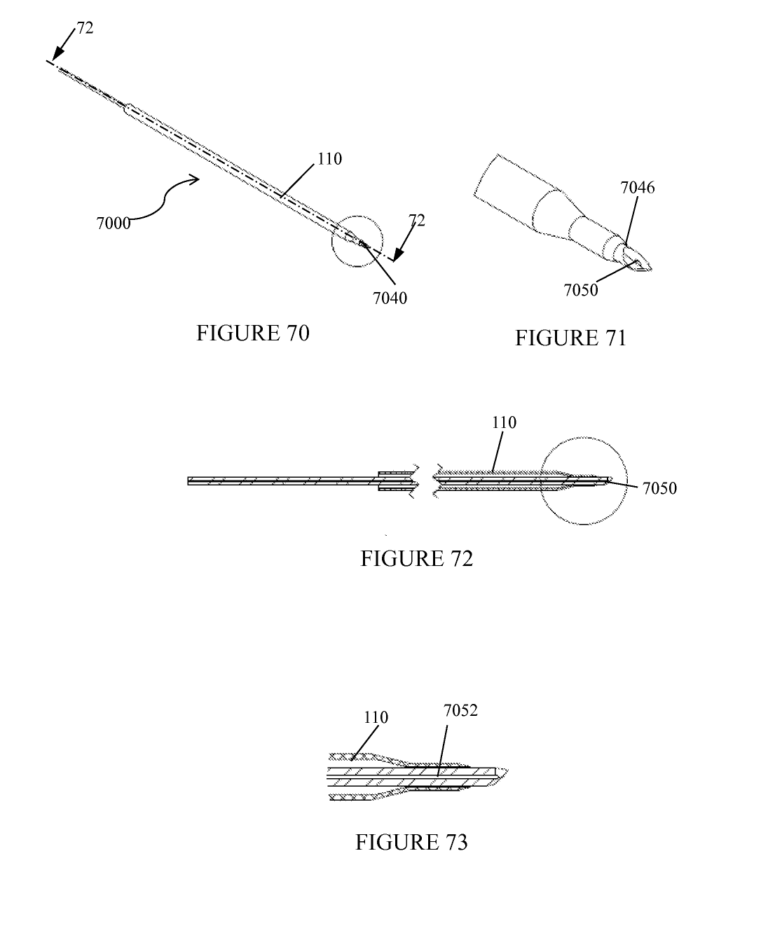

FIGS. 70 through 73 show a delivery device 7000 with a retractable member 7040. The retractable member 7040 may an opening 7050 disposed on a surface of a puncture member 7046. The opening 7050 may communicate with a channel 7052 that disposed along the length of the retractable member 7040. The channel may be configured to deliver a solution for dilating and/or expanding the delivery channel. The solution may include but is not limited to carbon dioxide and/or saline solution.

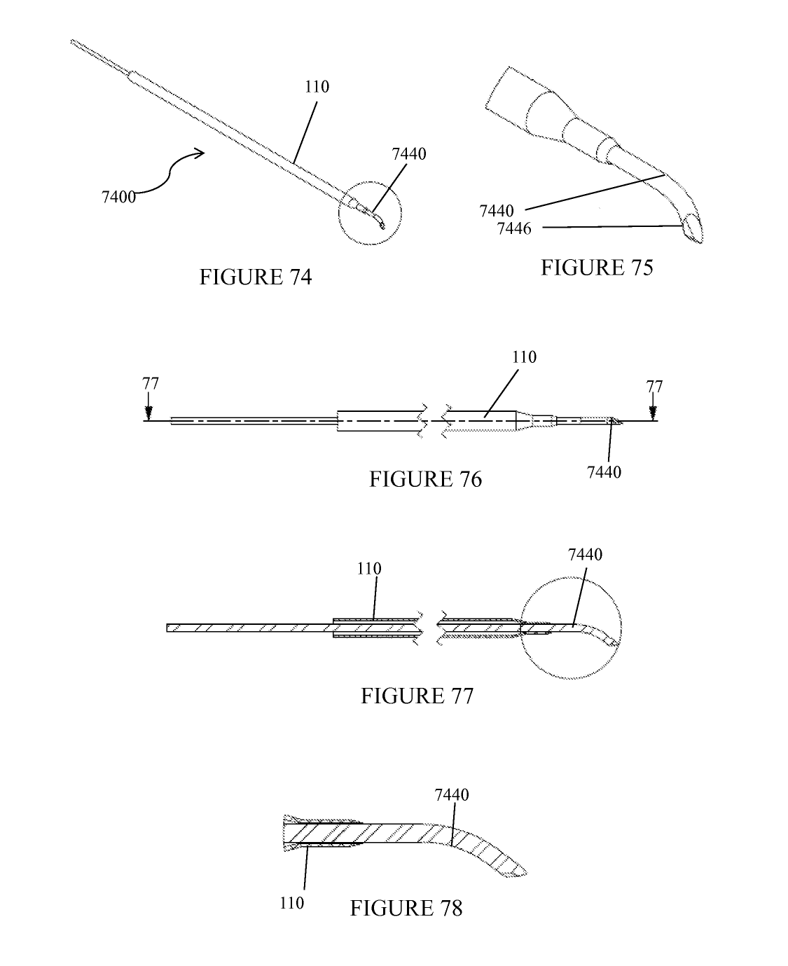

FIGS. 74 through 80 show an example of a delivery device 7400 with a retractable member 7440. The retractable member 7440 may include a puncture member 7446 disposed at an end. The puncture member 7446 may be like puncture member 5940 or may be according to different embodiments.

In some embodiments, at least a portion of the retractable member 7440 that includes the puncture member 7446 may be configured to be spring loaded so that it may be bend in a predetermined direction when extended past the delivery body 110. The retractable member 7440 may be configured to be retracted and rotated so that a plurality of delivery channels may be formed that converge at the delivery throat of the delivery body 110, thereby better distributing and dispersing a therapeutic agent to the treatment site. FIGS. 74 through 78 show the retractable member 7440 in one position, and FIGS. 79 and 80 show the retractable member 7440 in a different position (after the retractable member 7440 has been retracted into the delivery body 110 and rotated).

### Delivery Devices & Systems

It will be understood that the delivery devices according to the embodiments may be implanted into a patient with use of a delivery system. The delivery devices may be a part of the delivery system. The delivery system may be any known delivery system configured to advance and control the advancement of the delivery device to the treatment site. An example of the delivery system is shown in FIGS. 81 through 83.

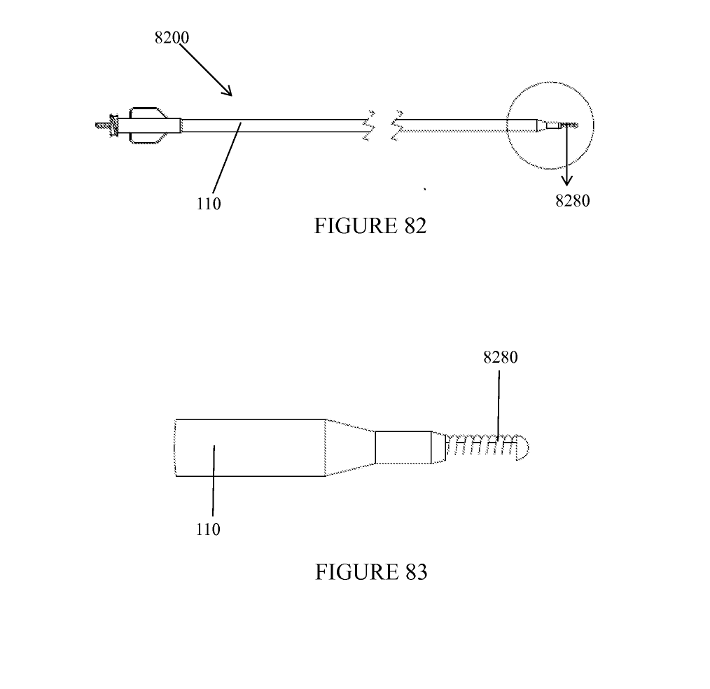

As shown in FIG. 81, the delivery systems and/or devices 8100 may be configured so that a clinician 8110 may percutaneously advance a delivery device to a treatment site 8120, for example, by using a micromanipulator (not shown). As shown in FIG. 82, a delivery system 8200 may include a guide wire 8280. The guidewire 8280 may be configured to be movable with respect to a delivery body 110 and may be configured to advance the delivery body 110 to the treatment site. The guidewire 8280 may also be configured to advance other elements of the disclosed delivery devices, for example, a retractable member. It will be understood that the delivery systems are not limited to those shown in the figures and the delivery devices may be used with other delivery systems. It will also be understood that a clinician may use another delivery system to control the operation of the disclosed delivery devices.

In some embodiments, the delivery device may be sterilized. In some embodiments, one, some, or all parts of the delivery device may be reused. In further embodiments, one, some, or all parts of the delivery may be disposable. In further embodiments, the delivery device may be a single, use device.

In some embodiments, the delivery device may be part of a kit. In some embodiments, the kit may include the delivery body and/or the retractable member. In some embodiments, the kit may include a guidewire.

In some embodiments, the delivery device may be preloaded with the therapeutic agent. In other embodiments, the agent may be loaded into the delivery device via a cartridge.

While the disclosure has been described in detail with reference to exemplary embodiments, those skilled in the art will appreciate that various modifications and substitutions can be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed:

1. A device for delivering an agent to a treatment site, comprising:
a body having a first end, an opposing second end and a length between the first end and the second end, the body including a channel between the first end and a second end, the channel including at least one opening configured to deliver the agent, the body including a first section and a second section, a diameter of the first section being larger than a diameter of the second section, a portion of the second section being parallel with the first section, the diameter of the inner channel corresponding to the diameter of the body; and
a retractable member having a first end, an opposing second end, and a length between the first end and the second end, the retractable member including at least one puncture member configured to penetrate tissue to create a delivery channel at the treatment site, the at least one puncture member being disposed at the second end, the retractable member being configured to move relative to the body;
wherein the retractable member is configured to move with respect to the body; and
wherein the body is configured to deliver the agent when the retractable member including the puncture member is retracted within the body.

2. The device of claim 1, wherein the second section includes a first portion and a second portion, the second portion being parallel with the first section.

3. The device of claim 2, wherein the second portion is longer than the first portion.

4. The device of claim 2, wherein the first portion is longer than the second portion.

5. The device of claim 1, wherein the opening is perpendicular to the length of the body.

6. The device of claim 1, wherein the opening is transverse to the length of the body.

7. The device of claim 1, wherein the retractable member includes a plurality of ports at least partially disposed on the second section.

8. The device of claim 1, wherein the first portion has a diameter that tapers from the first section to the second portion.

9. The device of claim 1, wherein the retractable member has a diameter configured to seal the opening of the delivery body.

10. The device of claim 1, wherein the puncture member includes at least one beveled edge.

11. The device of claim 1, wherein the puncture member includes a cutting edge.

12. A device for delivering an agent to a treatment site, comprising:
a body having a first end, an opposing second end and a length between the first end and the second end, the body including a channel between the first end and a second end, the channel including at least one opening configured to deliver the agent, the body including a first section and a second section, the second section including a first portion and a second portion, a diameter of the first section being larger than a diameter of the second section, the second portion being parallel with the first section, the diameter of the inner channel corresponding to the diameter of the body; and
a retractable member having a first end, an opposing second end, and a length between the first end and the second end, the retractable member including a first puncture member configured to penetrate tissue to create a delivery channel at the treatment site, the first puncture member being disposed at the second end, the retractable member being configured to move relative to the body;
wherein the retractable member is configured to move between at least a first position and a second position, the first position corresponding to when a portion of the retractable member is disposed within the channel, the second position corresponding to when at least the portion of the retractable member is retracted within the body, and
wherein the body is configured to deliver the agent when the retractable member is disposed at the second position.

13. The device of claim 12, wherein the body includes a plurality of extending members disposed at the second end, the body being configured to move between a tension state and an untension state.

14. The device of claim 12, wherein the extending members are tapered.

15. The device of claim 13, wherein the retractable member includes a plurality of protruding members and curved surfaces between the protruding members, the protruding members being configured to overlap the extending members and the curved surfaces being configured to move under the protruding members.

16. The device of claim 15, wherein the retractable member is configured to rotate with respect to the body.

17. The device of claim 12, the device further comprising:
a second puncture member, the second puncture member being disposed along the length of the body.

18. The device of claim 12, wherein the second puncture member includes a cutting edge configured to cut tissue and a tissue removal port configured to retain and remove the tissue.

19. The device of claim 12, wherein the retractable member includes an opening disposed at the puncture member and an inner channel that disposed along the length, the inner channel communicating with the opening.

20. The device of claim 12, wherein a portion of the retractable member is flexible.
